(19) United States
(12) Patent Application Publication
(10) Pub. No.: US 2023/0232903 A1
(43) Pub. Date: Jul. 27, 2023

(54) Portable Liquid Vaporizer

(71) Applicant: Cortney SMITH, Las Vegas, NV (US)

(72) Inventors: Cortney Smith, Las Vegas, NV (US); Josh Wang, Las Vegas, NV (US); Markus Haller, Bad Neuenahr-Ahrweiler (DE); Christian Hauptman, Bad Neuenahr-Ahrweiler (DE); Alexander Wegener, Bad Neuenahr-Ahrweiler (DE)

(21) Appl. No.: 18/007,170

(22) PCT Filed: Aug. 19, 2021
(86) PCT No.: PCT/IB2021/057634

§ 371 Date: Jan. 27, 2023

Related U.S. Application Data
(60) Provisional application No. 63/067,523, filed on Aug. 19, 2020.

(51) Int. Cl. A24F 40/48; A24F 40/10; A24F 7/00; A24D 3/17; A24F 40/30; A24F 40/51; A24F 40/57; A24F 40/53; A24F 40/42

(52) U.S. Cl. CPC A24F 40/48; A24F 40/10; A24F 7/00; A24D 3/17; A24F 40/30; A24F 40/51; A24F 40/57; A24F 40/53; A24F 40/42

(57) ABSTRACT

A portable liquid vaporizer (1) comprises a reservoir (10) for holding a liquid to be vaporized; a heater (12) for vaporizing the liquid; a mouthpiece (3) for withdrawing the vapor; and an air path (30) extending through the vaporizer (1), comprising a heating section (31) and a cooling section (32), wherein the heating section (31) extends along the heater (12) and the cooling section (32) extends directly downstream from the heater (12) to the mouthpiece (3).

The present invention lies in the field of vaporizers. More particular, the invention relates to a portable liquid vaporizer comprising a reservoir for holding a liquid to be vaporized, a heater for vaporizing the liquid, a mouthpiece for withdrawal of the vapor, and an air path extending through the vaporizer. The air path comprises a heating section that extends along the heater. During normal operation, a user will place its lips on the mouthpiece and inhale through the device.

The air path refers to the path that air and vapor must travel through the vaporizer, i.e. between an inlet and an outlet (=mouthpiece) of the device. In liquid vaporizers, this air path is typically between 10 mm and 15 mm because the pod/heater is located at the top of the device (adjacent to the mouthpiece).

Vaporizers of this kind are for example known from Chinese utility model 204217916. The electronic cigarette disclosed therein comprises a cylindrical shell, an atomization assembly mounting seat, an atomization assembly, a liquid storage member, a power supply assembly, an end cover and a mouthpiece cover. The mouthpiece cover and the end cover are respectively sealed on the two ends of the cylindrical shell. The atomization assembly mounting seat, the atomization assembly, the liquid storage member, and the power supply assembly are all installed inside the cylindrical shell, wherein the atomization assembly mounting seat is arranged at a central position of the cylindrical shell. The atomization assembly is arranged on the atomization assembly mounting seat and is between the atomization assembly mounting seat and the mouthpiece cover. The liquid storage member is arranged on a periphery of the atomization assembly. The power supply assembly is arranged between the atomization assembly mounting seat and the end cover, and is electrically connected to the atomization assembly. The atomization assembly includes a liquid guiding component that is wound around a heating element. The heating element comprises an elongated body and at least one protrusion, wherein the at least one protrusion is provided on the elongated body and is at least partially embedded in the liquid guiding component.

The benefit to placing the heater (atomization assembly) at the top as in the electronic cigarette disclosed in CN 204217916 U is a lower cost and lesser complexity. While financially desirable, this configuration is associated with the disadvantage that the vapor may still be very hot when it comes into contact with the user's lips with larger droplets of partially vaporized liquid being inhaled. This may irritate user experience and even lead to burn injuries.

A problem to be solved by the present invention is hence the provision of a vaporizer, which overcomes said disadvantage, and in particular which safeguards that the vapor has a comfortable temperature when coming into contact with a user's lips.

This problem is solved by a vaporizer of the aforementioned type comprising a cooling section as disclosed herein.

Further objectives become apparent from the following description and especially from the described advantages.

According to a first aspect of the present invention, a vaporizer of the aforementioned type comprises a cooling section extending directly downstream from the heater until the mouthpiece. The cooling section is part of the air path and extends directly downstream from the heater to the mouthpiece. The phrase “directly downstream” implies that the cooling section begins immediately after the heater/heating section, whereby positional information indicated herein corresponds to the direction of the flow of the vapor through the vaporizer (during normal use). That is, a position Y downstream from position X means that the vapor passes position Y “after” position X, i.e. that the vapor first passes position X and then position Y. The reservoir, the heater and the air path (including the cooling section) and, if present, further componentry of the vaporizer, are preferably encased by a housing. The housing may be made of aluminum and extend circumferentially around the componentry, defining a lumen with an open top end that is enclosed by the mouthpiece and an open bottom end that is enclosed by a bottom cap. The mouthpiece and the bottom cap are preferably made from plastic.

The housing may further comprise at least one window, preferably two windows located on opposite sides of the housing. The window(s) allow a user to assess the amount and color of the liquid in the reservoir. The color of the liquid can be used as an indicator of the liquid's quality. The housing may further comprise an array of LEDS, which may be used as a tool to communicate information with the user.

The cooling section preferably has an effective length that is sufficient for allowing the vapor to cool down and leave the mouthpiece at 40° C. or less above environmental temperature, preferably 35° C. or less above environmental temperature, more preferably 30° C. or less above environmental temperature and most preferably 20° C. or less above environmental temperature upon withdrawal of the vapor. The environmental temperature is the temperature of the surrounding, where the vaporizer is used. Since the vapor temperature is also affected by the environment, the cooling capacity is defined in relation to the environmental temperature. Preferably, the cooling section preferably has an effective length as defined above, when determined at an environmental temperature of 20° C.

The first aspect of the present invention is based on the innovation of the inventors that vapor to be withdrawn is cooled down to comfortable temperatures using a cooling section based on an extended air path. In this context, the inventors found that an extended air path combines an advantageous property profile. An extended air path can be easily manufactured and can be integrated into the vaporizer in a cost-efficient manner. There are no additional installations such as a heat sink or the like required. Moreover, by extending the air path, it is not necessary to increase the surface area/volume-ratio of the air path. This means, that the size (diameter) of the air path can be selected to best fit the user experience.

Whether an air path has a cooling section with an effective length as defined herein can be routinely determined using the following testing procedure: Insert a thermocouple to the mouthpiece of the vaporizer, attach a vacuum pump over the thermocouple to the mouthpiece and, optionally, test the assembly for leakage. If applicable, adjust the vaporizer to maximum output settings. Rest the assembly until the thermocouple and the environmental temperature reaches equilibrium. Set the vacuum pump to 80 mL/s. Using the vacuum pump, draw vapor for 10 seconds and then rest for 30 seconds. Repeat this step for 10 times. For each draw, measure temperature and note temperature peaks on each draw. The averaged temperature peaks define the temperature at which the vapor left the mouthpiece.

In a preferred embodiment of the present invention, the cooling section has an effective length that is at least 50%, preferably at least 60%, more preferably at least 70%, most preferably at least 80% of the length of the vaporizer. The length of the vaporizer defines its largest extension. By occupying a great length relative to the length of the vaporizer, the vapor can be efficiently cooled down, yet the device remains compact. Preferably, the cooling section has an effective length that is at least 50 mm, preferably at least 60 mm, more preferably at least 65 mm and most preferably at least 70 mm.

In a further preferred embodiment of the present invention, when the mouthpiece defines the top, the heater is located in the lower half, preferably in the lower third, more preferably in the lower quarter, of the vaporizer. Consequently, the distance between the heating section and the mouthpiece, i.e. the distance of the cooling section, is relatively large. With such configuration it is not necessary to arrange the cooling section in serpentines or the like to cover a sufficient distance.

In a further preferred embodiment of the present invention, a power source is arranged between the heater and the mouthpiece. This means, the power source spaces apart and thereby defines a minimum distance of the cooling section. As an advantage of this configuration, the vaporizer can be made small, yet provide a long cooling section. In particular, it is not necessary to use an external elongation such as a tube.

Another embodiment of the invention provides that the longitudinal extension of the cooling section is offset from the longitudinal axis of the vaporizer. When the power source is between the heater and the mouthpiece, the power source may occupy a large volume. It is thus advantageous that the cooling section is routed along and close to the housing. Moreover, at the mouthpiece, the air path preferably extends coaxial to the longitudinal length of the vaporizer. In that the cooling section extends partly offset and partly coaxial to the longitudinal length of the vaporizer, an additional length increase of the cooling section is achieved.

According to another embodiment of the present invention, the air path extends from the bottom of the vaporizer to the mouthpiece at the top of the vaporizer. Moreover, the air path preferably comes in contact with the liquid from the reservoir directly upstream from the heating section.

A second aspect of the present invention relates to a portable liquid vaporizer, preferably the portable liquid vaporizer as described above, comprising a reservoir for holding a liquid to be vaporized, a heater for vaporizing the liquid, a mouthpiece for withdrawal of the vapor, and an air path extending through the vaporizer, comprising a heating section and a cooling section, wherein the heating section extends along the heater and the cooling section extends directly downstream from the heater to the mouthpiece, wherein the cooling section comprises an insert for absorbing heat, filtering the vapor, catching droplets and/or flavoring the vapor.

In various embodiments, the insert is removable (as opposed to a permanent installation). An exemplary removable insert comprises a mesh-like or sponge-like body that fits in the air path so that the vapor passes there through. In this way, the vapor can be filtered and droplets can be caught and retained by the body. Moreover, in some embodiments it is preferred that the mesh-like or sponge-like body is flavored. In this way, in addition to the effects achieved with the body as such, the flavor contained in the body is transferred to the vapor, thereby affecting the user experience.

In other embodiments, the insert may be permanently installed. An example in this regard is an installation configured to guide the vapor helically through the cooling section. In this way, the length of the cooling section can be increased.

A third aspect of the present invention pertains to a portable liquid vaporizer, preferably the portable liquid vaporizer as described herein, comprising a reservoir for holding a liquid to be vaporized, a heater for vaporizing the liquid, a mouthpiece for withdrawal of the vapor, an air path extending through the vaporizer, comprising a heating section and a cooling section, wherein the heating section extends along the heater and the cooling section extends directly downstream from the heater to the mouthpiece, a control unit for controlling the heater, and one or more, preferably one or two, temperature sensor(s) for determining the temperature of the vaporized liquid in the cooling section, wherein, when the temperature sensor(s) determine(s) a temperature exceeding a predetermined threshold, the control unit is configured to reduce or stop the heating power of the heater.

The temperature sensor(s) is/are preferably located in the cooling section, adjacent to the heater and/or adjacent to the mouthpiece. A temperature sensor adjacent to the heater enables to accurately determine the temperature of the heater. This guarantees that the device will not produce vapor at dangerous temperatures where outgassing can occur with the materials used. Additionally, controlling the temperature adjacent to the heater may guarantee a more consistent temperature at the mouthpiece of the device. A temperature sensor adjacent to the mouthpiece provides a good estimate and control of the temperature of the inhaled vapor. This guarantees that the user will always experience a controlled vapor temperature, and contributes to preventing a user to inhale too hot vapor. For instance, when the control unit determines that the temperature detected by the temperature sensor is at or above a predetermined threshold, the temperature of the heater is lowered, or the heater is switched off.

According to a further embodiment of the present invention, the vaporizer further comprises a control unit for controlling the heater, and a flow detector for detecting whether a user inhales through the mouthpiece. The term “flow detector” as used herein refers to a detector that is capable to detect whether or not flow occurs through the vaporizer. By means of the flow detector, upon inhalation, the flow detector detects that a user inhales through the mouthpiece, and the control unit switches on or increases the heating power of the heater. In this way, liquid is only heated when needed. Power consumption can thereby be decreased and battery life can be increased. Moreover, vapor is prevented from escaping unused into the environment. In addition, prolonged exposure to heat may favor side and/or decomposition reactions, which could be avoided by the described embodiment.

The flow detector may be selected from the group consisting of differential pressure sensors, capacitive air flow sensors, spinning fans/turbines, moving flap-type sensors, temperature sensors and thermal flow sensors. These detectors can be easily integrated into the portable vaporizer. The concept by which the aforementioned flow detectors work is known to a person skilled in the art and briefly described in the following.

Differential pressure sensors: A small sensor that measures pressure at 2 locations—a significant difference in those measurements signifies flow. Output: Occurrence and/or intensity of flow.

Capacitive air flow sensors: A small diaphragm flexes when pressure drops on one side of it as a result of flow. The change in geometry causes a change in capacitance which signifies flow. Output: Occurrence.

Spinning fans/turbines: A small fan is placed in the air path. User inhalation spins the fan which signifies flow. Output: Occurrence and intensity.

Moving flap-type sensors: Similar to a spinning fan/turbine. User inhalation pushes the flap which signifies flow. Output: Occurrence and intensity.

Temperature sensors: Only applicable when there is a change in temperature—a significant difference in temperature between the initial measurement and the final measurement signifies flow. Output: Occurrence and intensity.

Thermal flow sensors: A small heater is positioned between an upstream and a downstream temperature sensor. User inhalation heats the downstream sensor. The difference between the upstream and downstream sensor signifies flow. Output: Occurrence and intensity.

Preferably, the flow detector is a diaphragm pressure sensor.

Another preferred embodiment of the present invention provides a vaporizer as described herein, further comprising a flow rate sensor for determining the flow rate through the air path. The flow rate sensor may be selected from the group consisting of differential pressure sensors, spinning fans/turbines, moving flap-type sensors, temperature sensors and thermal flow sensors. The flow rate sensor allows for calculation of active compound dosage.

In particular, a dosage can be calculated based on the flow rate, heater temperature, heating time and percentage of active agents in the liquid. It is therefore preferred that the control unit is further configured to estimate a dosage of one or more active agents withdrawn from the vaporizer based on a mathematical model, wherein the mathematical model relates heating time, temperature of the heater, the flow rate and amount of the one or more active agents contained in the liquid to the dosage of the one or more active agents withdrawn from the vaporizer. The dosage estimation provides the basis for dosage control. Accordingly, it is preferred that the control unit is further configured to control the dosage of the one or more active agents withdrawn from the vaporizer. In respect of suitable mathematical models in this regard, two are explained in the following. The first model to calculate dosage is more accurate than the second model but the second model is more cost-efficient and more simple. Based on the explanations, a person of ordinary skills in the art can readily construct modified models. Hence, the present invention is not limited to the following models.

The calculation and display of dosage information may occur on the vaporizer itself (for example via LEDs as described herein) and/or on an application such as a mobile APP, associated with the vaporizer.

The vaporizer will take in data from the pod about the liquid contained (either user input or automatically via data stored on the pod or a QR code/serial number to call data from an online server), data from the temperature sensor adjacent to the heater, and data from the draw (flow) sensor.

The term “pod” as used herein includes any reservoir containing liquid and a heating element. In particular, the vaporizer disclosed herein include vaporizers which are also known as pod-based vaporizers and vaporizers which are also known as cartridge-based vaporizers. Both work very similarly: a pod or cartridge containing a heating element and a liquid reservoir mates with a battery. Pods mate using proprietary mating surfaces to the battery thus being usually incompatible with any other non-proprietary pod. Cartridges mate using common mating surfaces to the battery thus so long as a cartridge has that common mating surface, it is compatible with the battery. The terms “pod-based” and “cartridge-based” are used to differentiate between the aforementioned hardware formats.

The pod will give the amount of oil, the type of oil, and its active ingredient(s) (e.g. cannabinoid profile) percentages.

The temperature sensor adjacent to the heater will give the vapor temperature exiting the pod.

The draw sensor will give the time duration of the draw.

One assumption that is made is that each type of extract (liquid) behaves similarly to itself when vaporized, namely in its viscosity, density, specific heat, and so on such that most commonly sold distillate behaves like an average distillate, most commonly sold CO2 oil behaves like an average CO2 oil, most commonly sold live resin behaves like an average live resin, and so on. Tests are made with each type of liquid. For example, 5 different distillates, 5 different BHOs, 5 different live rosins, and so on are tested and then the empirical data for the distillates, the BHOs, the live rosins, and so on are averaged. The number of types of extracts tested for ideally capture a majority of the types of extracts intended to be filled in the pods.

Preferably, a larger sample size and every batch of extract type is tested. Because oils sold on the market are typically cut with propylene glycol, vegetable glycerin, or terpene extracts with the amounts undisclosed to make the extract itself behave better during heating and vaporization, no two extracts behave the same.

It is important to note that the type of extract influences its dosing because while most extracts intended for vaporizer usage are decarboxylated during the extraction process and others are not and this can depend on the supplier. Decarboxylated extracts have most if not all their THC/CBD content ready for vaporization and consumption. Non-decarboxylated extracts must be decarboxylated by the heater—the time that extract experiences under heat is remarkably short (less than 1 second), so not all of the THC/CBD content will be available to the user. In the most ideal case, not only different types of common extracts, but also the common decarboxylated/non-decarboxylated variants of those extracts are tested.

The dosage will be driven by an empirically tested data table. Along the x-axis is vapor temperature above environmental temperature and along the y-axis is draw duration.

### EXAMPLE

THC Production Rate (Distillate); Units [mg/s]

| | | | Temperature Range (F) above | | | | | | |
|---|---|---|---|---|---|---|---|---|---|
| | | | Environment | | | | | | |
| | | | 0 | 21 | 41 | 61 | 81 | 101 | Floor |
| | | | 20 | 40 | 60 | 80 | 100 | 120 | Ceiling |
| Time | 0 | 6 | | | | | | | |
| Range | 7 | 12 | | | | | | | |
| (sec) | Floor | Ceiling | | | | | | | |

The above table may be duplicated for the type of extract (distillate, BHO, CO2, and so on) and the cannabinoid (THC, CBD).

To establish a reasonable range of values for the x-axis, the vaporizer is tested at each of its heater power settings to determine a relationship between heater power and vapor temperature. The temperature of the heater of the vaporizer may be controllable via wire resistance feedback, but this is less accurate than an actual thermocouple placed directly on the heater. Additionally, this control is much less accurate as heat-up time is relatively fast and there is a small separation distance between the heater and the temperature sensor.

Additional variables would be multiplied by the values in the table above. These additional variables would dictate modifications such as limiting the total pod dose such that it does not exceed the theoretical amount, accounting for decomposition effects in a pod over time, accounting for whether the extract is decarboxylated or not, and so on. These additional variables may also by their own data tables, dependent on other conditions.

An alternative dosage calculation method is possible based on the assumption that each draw reduces the total oil mass by some consistent amount. This alternative method makes further assumptions for the sake of simplicity and only requires a relationship between draw time and an average total oil mass loss. It posits that at some heater power setting, each second of vaping will reduce the total liquid mass by some amount. That amount converts completely to vapor which the user inhales. The vaporized amount can then be multiplied by the percentage THC/CBD to calculate the THC/CBD content of the draw. This method would disregard decomposition effects, un-decarboxylated extract, and so on and would not require extensive testing of different types of liquids as is the case with the first method.

A fourth aspect of the present invention relates to a portable liquid vaporizer, preferably a portable liquid vaporizer as described herein, comprising a reservoir for holding a liquid to be vaporized, a heater for vaporizing the liquid, a mouthpiece for withdrawing the vapor, an air path extending through the vaporizer, the air path comprising a heating section and a cooling section, wherein the heating section extends along the heater and the cooling section extends directly downstream from the heater to the mouthpiece, wherein the heater is at least partly surrounded by a thermally stable plastic or an insulation.

In one preferred embodiment of the present invention, the thermally stable plastic is made from PEEK or PCTG. It has been found that these plastics withstand the temperature of the heater. Using these plastics, an insulation that separates the heater from plastic parts of the vaporizer is not needed. However, this does not preclude the use of an insulation. If an insulation is used, it is preferably made of ceramics.

A fifth aspect of the present invention relates to a portable liquid vaporizer, preferably a portable liquid vaporizer as described herein, comprising a reservoir for holding a liquid to be vaporized, a heater for vaporizing the liquid, a mouthpiece for withdrawing the vapor, an air path extending through the vaporizer, comprising a heating section and a cooling section, wherein the heating section extends along the heater and the cooling section extends directly downstream from the heater to the mouthpiece, wherein the vaporizer is configured to prevent unauthorized manipulation thereof.

The fifth aspect relates to tamper proofing. Tamper proofing refers to the prevention of unauthorized manipulation, such as oil filling into the pods, without noticeable, significant damage to any part of the device and to the prevention of unauthorized pod use by the end user. According to a preferred example in this regard, the reservoir is embedded in a tamper proof pod section so that the reservoir cannot be accessed without damage or permanent deformation to the pod section. The pod section comprises the reservoir and the heater. By embedding the reservoir in a tamper proof pod, the reservoir is prevented from manipulation and/or unauthorized re-filling.

According to a further embodiment, a power source may be embedded in a tamper proof battery section. The tamper proof battery section prevents manipulation of the battery, and avoids that the vaporizer is equipped with unofficial third party batteries.

The vaporizer preferably comprises a replaceable pod section and is configured to authenticate a certified pod section. Preferably, the authentication is based on verifying a data key stored on the pod section. For this purpose, the pod section may comprise a data storage such as an EEPROM, and the vaporizer checks the data stored in the data storage. Only when the correct data are found, the vaporizer can be used. In this way, unauthorized pods cannot be used with the vaporizer

According to a further preferred embodiment of the present invention, the vaporizer comprises a control unit for controlling the vaporizer, in particular the heater depending on data stored on the pod section. This means, the vaporizer/control unit may not be only configured to check whether a certified pod section is included in the device, but may also read further data from the pod section such as liquid data, and adjust the behaviour of the vaporizer to the data. This allows the vaporizer's functioning (such as the heating temperature, the heating profile and/or the heating time) to be tailored to the liquid contained in the pod section. Other data that may be included on the pod section can be the store retailer, manufacturer, and oil filler data.

According to a preferred embodiment of the present invention, the liquid is cannabis concentrate, such as cannabis oil or cannabis wax. Cannabis is a flowering plant often consumed in its ‘loose-leaf’ or flower form or in a variety of liquid, concentrated forms and purchased legally in many countries at dispensaries. Two active agents are of medical interest: delta-9-tetrahydrocannabinol (“THC”) and cannabidiol (“CBD”), which belong to the class of cannabinoids. THC is the psychoactive component within the plant, causing the “high” commonly associated with its use. CBD is a form of THC but acts as a pain relieving rather than a psychoactive agent. Both THC and CBD appear in precursor forms tetrahydrocannabinolic acid (“THCA”) and cannabidiolic acid (“CBDA”), respectively, and are converted to their active forms upon heating known as decarboxylation or activation. The vaporizer heats the liquid cannabis concentrate, generating vapor that contains THC and/or CBD. Users inhale through the device to simultaneously withdraw and consume the vapor.

Cannabis oil is a concentrated extract obtained by extraction of the dried flowers or leaves of the cannabis plant. Chemically, it is not an oil, but derives its name from its sticky and oily appearance. Cannabis wax is a concentrated extract obtained by extraction with an extracting solvent. The purpose of producing cannabis oil and wax is to make cannabinoids available in a highly concentrated form. The extracts that typically are used are made using extracting solvents, like butane (known as “BHO”) or carbon dioxide (“CO2 oil”), or made using heat and pressure (“Rosin”).

The vaporizers of the first, second, third, fourth and fifth aspects have many features in common with each other, and each of the features and embodiments described in regard of any aspect shall be understood to define corresponding features and embodiments of all other aspects.

A sixth aspect of the present invention pertains to a mouthpiece for a portable liquid vaporizer, wherein the mouthpiece comprises a cooling section that is configured to extend an internal air path of the vaporizer, wherein the cooling section is configured as described herein. The mouthpiece serves as an extension of the internal air path of vaporizers having no cooling section or which cooling section is too short to achieve sufficient cooling of the vapor. The mouthpiece may take the form of a rigid tube or a flexible tube defining an inner lumen with a first open end to be coupled to the vaporizer and a second open end for withdrawing the vapor. It may be made of a variety of materials, preferably from glass or from plastic. The mouthpiece may comprise a coupling element at the first open end so that the mouthpiece can be firmly coupled to the vaporizer. The mouthpiece may further comprise a sealing element at the first open end to seal the mouthpiece against the vaporizer.

The functionalities of the vaporizers and the mouthpiece of the present invention as disclosed herein can be translated into corresponding uses and methods, which are encompassed by the present invention.

These and other aspects and embodiments of the invention will become apparent from and elucidated with reference to the embodiments described hereinafter taken in conjunction with the accompanying drawings. Further advantages will be apparent to those of ordinary skill in the art upon reading and understanding the drawings and the description.

The drawings may show features that are not recited in the claims to improve their understanding. These features should be understood as merely optional unless dictated otherwise by context. The individual features of each aspect or embodiment may each be combined with any or all features of other aspects or embodiments.

In the following drawings:

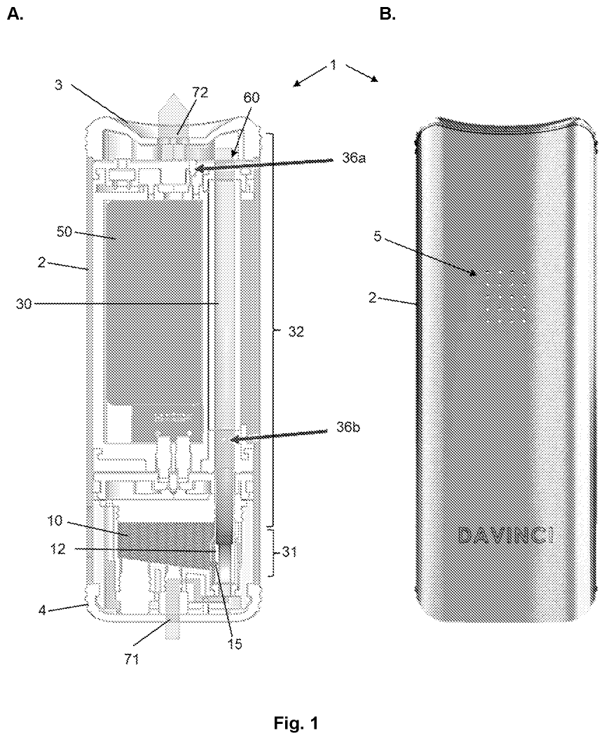

FIG. 1 shows a portable liquid vaporizer according to embodiments of the first, second and third aspects of the present invention.

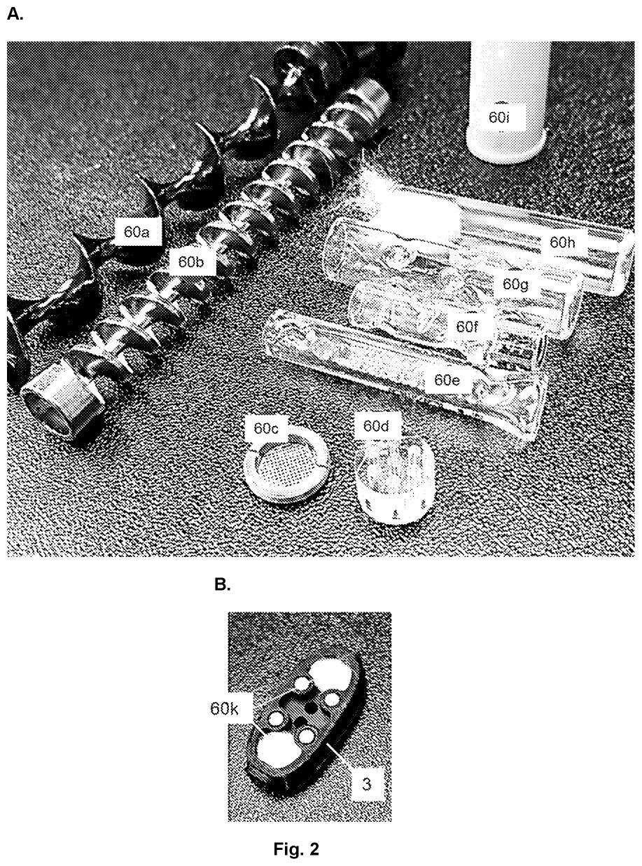

FIG. 2 shows different inserts for use with a vaporizer according to embodiments of the second aspect of the present invention.

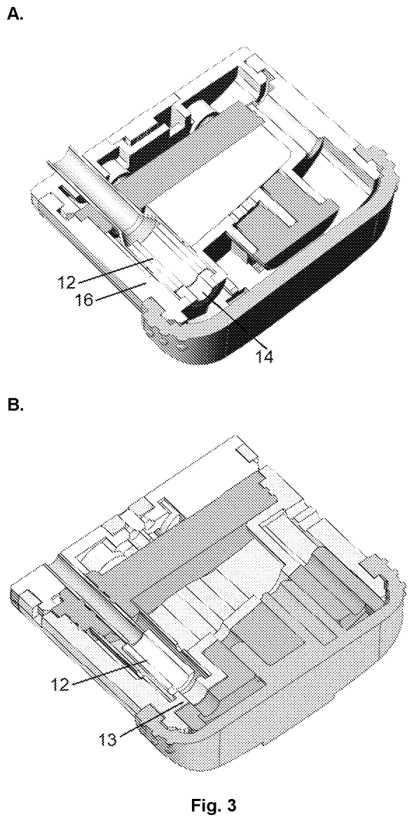

FIG. 3 shows a detail of the vaporizer according to embodiments of the fourth aspect of the present invention in a sectional view.

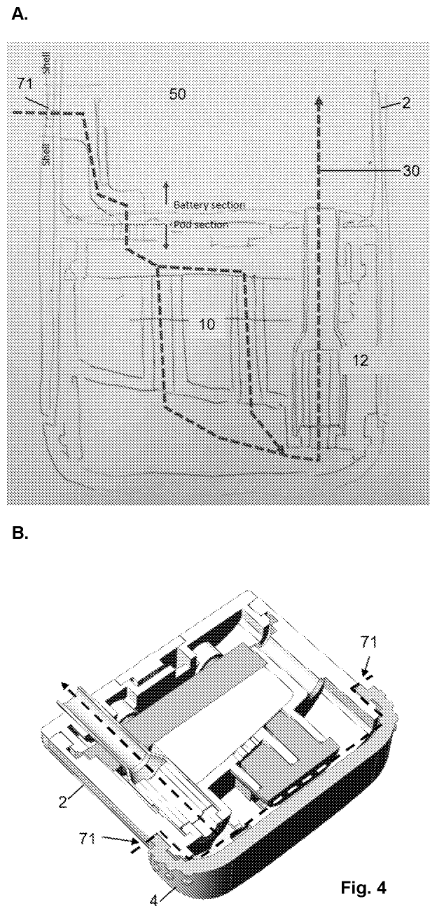

FIG. 4 shows air path routes of vaporizers according to embodiments of the present invention.

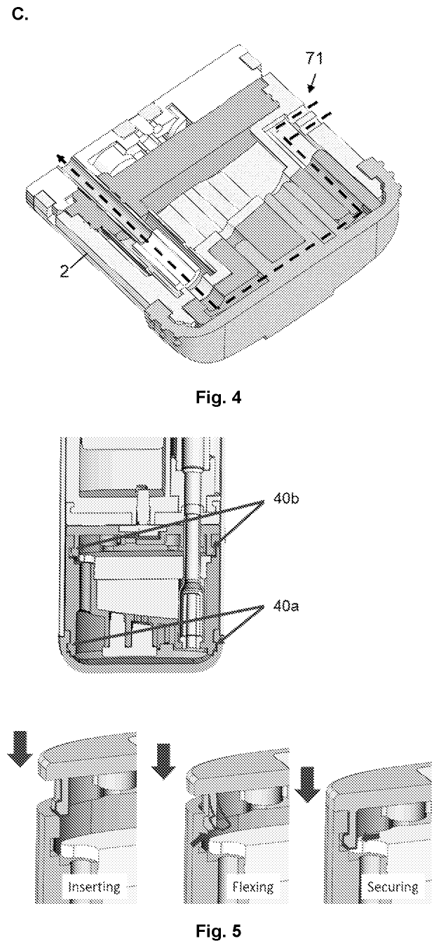

FIG. 5 shows a portable liquid vaporizer according to an embodiment of the fifth aspect of the present invention.

FIG. 1 shows a portable liquid vaporizer 1 according to a preferred embodiment of the first aspect of the present invention. The vaporizer 1 (also referred to herein as the device 1) is for use with cannabis in liquid (including oily or waxy) form. As can be seen in FIG. 1A, a reservoir 10 is filled with a liquid to be vaporized. A heater 12 heats the liquid, transferring it into the vapor phase. An air path 30 extends through the vaporizer 1. In the shown embodiment, the liquid enters the air path 30 through a slit-shaped inlet 15. Inlet 15 provides an entry for the liquid into the heating section 31, where the liquid is vaporized. The vapor can be withdrawn from the vaporizer through a mouthpiece 3. During normal operation, a user will place its lips on the mouthpiece 3 and inhale through the device 1. Upon inhalation, the device 1 may detect the moving air (via a flow detector, not shown) and heats the liquid to vaporize it into vapor. The vapor is generated within a pod section, flows through a battery section and into the mouthpiece 3, then into the user's mouth. The pod section comprises the heater 12 and the reservoir 10 with the liquid. The battery section contains the power source 50, a section of the air path 30 that connects the pod section with the mouthpiece 3, and the mouthpiece 3 itself.

The vaporizer 1 comprises a housing 2, a mouthpiece 3 and a bottom cap 4 opposite of the mouthpiece 3, which encase the reservoir 10, the heater 12, the power source 50 and the air path 30. The mouthpiece 3 has a shape that conforms to the lips so that users purse their lips against the mouthpiece 3, rather than placing any part of the device 1 into their mouths. This reduces the amount of saliva that is left on the mouthpiece 3 and thus transferred when in a group-sharing setting.

The air path 30 comprises a heating section 31. The heating section 31 is defined as the section of the air path 30 that extends along the heater 12. In the present embodiment, the heating section 31 is encircled by the heater 31. The air path 30 further comprises a cooling section 32. The cooling section 32 is the part of the air path 30, which extends directly downstream from the heater 12 until the mouthpiece 3. This means, the cooling section 32 follows directly downstream from the heating section 31.

In the shown embodiment, an air inlet 71 is formed in the bottom cap 4. However, the air inlet can also be comprised in the housing 2, or between the housing 2 and the bottom cap 4 (as shown in FIG. 4). An air outlet 72 is contained in the mouthpiece 3. The air path 30 extends through the vaporizer 1, from the air inlet 71 until the air outlet 72 at its top (mouthpiece 3). By moving the heater 12 and the reservoir 10 towards the bottom of the vaporizer 1, and moving power source 50 (battery) towards the top, a long cooling section 32 is formed. It is seen that the heater 12 and a substantial part of the air path 30, including the heating section 31, may be routed around the power source 50, offset from the longitudinal axis of the vaporizer.

As shown in FIG. 1B, a group of LEDs 5 may be arranged in an ordered pattern on the housing 2 visibly to the users. The LEDs 5 allow to display information and are also referred to as display LEDs 5. Buttons (not visible) are placed on the housing 2 in order to switch the vaporizer 1 on and off, enter and navigate through a menu (if present). In the menu (if present), users may select different modes and input data. In the shown example, 20 LEDs are arranged in a pattern of 4×5 LEDs. However, there can be more or less LEDs in varying patterns. For instance, there can be a (one) row of 4 LEDs (not shown).

A portable liquid vaporizer 1 according to a preferred embodiment of the second aspect of the present invention is described with reference to FIGS. 1 and 2. The vaporizer 1 of the second aspect has many features in common with the vaporizer 1 of the first aspect, and each of the features and embodiments described in regard of the first aspect shall be understood to define corresponding features and embodiments of the second aspect. FIG. 1A schematically depict that an insert 60 may be included in the cooling section 32 of the device 1, at a location close to the mouthpiece 3 (in particular, where the arrow of reference sign 60 points to in FIG. 1A). FIG. 2 shows exemplary embodiments of such inserts 60. FIG. 2A shows a ceramic corkscrew insert 60a to absorb heat, a metal corkscrew insert 60b to absorb heat, a metal screen filter to catch stray droplets of oil (insert 60c), a glass filter to catch stray droplets of oil (insert 60d), a glass tube with pinched ends filled with glass beads to filter and cool vapor (insert 60e), a glass tube with cone structures to cool vapor (insert 60f), a larger glass tube with cone structure to cool vapor (insert 60g), a hollow glass tube with cotton insert for filtration (insert 60h), and a hollow ceramic tube with cotton insert for filtration (insert 60i).

Another insert for flavoring the vapor is depicted in FIG. 2B. The insert 60k is a sponge-like body that is placed on the mouthpiece 3 and allows users to modify their flavor experience by adding e.g. volatile terpenes to the air path outside of the pod. Users may benefit by purchasing flavorless oil pods or lower quality, cheaper oil pods and then augmenting the generated vapor with flavorful terpenes. On the higher price end, users could accentuate the flavor profiles of their favorite oils. Instead on the mouthpiece 3, the insert 60k can be located within the air path 30, close to the mouthpiece 3 (at a location where inserts 60a-60i can be located). For instance, an insert similar to the insert 60h, where a flavored piece of cotton is included in a hollow tube, may be inserted into the air path 30. To facilitate easy removal and cleaning of the insert, the hollow tube may be flanged at the top. Moreover, the hollow tube may be composed of an inner tube and an outer casing. The inner tube can be made from metal and may be perforated to allow flavors to evaporate. The outer casing may be made of metal and may seal the air path and hold the piece of cotton in place.

In FIG. 2, 60a-60f are components available on the market for different purposes, used as representative examples only. 60g to 60i are components from applicant's prototypes.

To allow an insert to be added to the air path 30, the mouthpiece 3 can be configured to be pivoted around a pivot axis away from the housing 2. Thereby, the flow path 30 can be accessed.

With further reference to FIG. 1, a portable liquid vaporizer 1 according to a preferred embodiment of the third aspect of the present invention is described next. The vaporizer 1 of the third aspect has many features in common with the vaporizer 1 of the first and the second aspect, and each of the features and embodiments described in regard of the first and second aspects shall be understood to define corresponding features and embodiments of the third aspect. As can be seen in FIG. 1A, there can be one or two temperature sensors 36a, 36b arranged in the air path 30. One temperature sensor 36a may be located close to the mouthpiece 3. The other temperature sensor 36b may be located close to the heater 12. In combination with a control unit (not shown) for controlling the heater, the vapor temperature can be controlled through a feedback loop between one or both temperature sensors 36a, 36b, the heater 12, and the control unit. Should the vapor temperature rise beyond either a preset value or a user-set value, the heater power may be reduced in order to regulate the vapor temperature. As temperature is preferably monitored continuously, this loop will occur whenever users inhale through the device and will continue through each inhalation.

Placing temperature sensor 36a close to the mouthpiece 3 enables a control of the temperature at that point—prior to the vapor entering the user's mouth. This guarantees that the user will always experience a controlled vapor temperature. However, as this sensor location is relatively far from the heater 12 and the vapor will naturally cool down as it travels upwards, the actual effectiveness of this sensor location may be questionable.

Placing temperature sensor 36b close to the heater 12 enables the control of the temperature at that point—immediately upon exiting the pod where the vapor is close to its hottest. This guarantees that the device 1 will not produce vapor at dangerous temperatures where outgassing can occur with the plastics and metals within the air path. Additionally, controlling at this sensor location may guarantee a more consistent temperature at the outlet of the device 1. However, as this sensor location is quite close to the heater 12, users may cause a shutdown through overheating the sensor 36b by rapidly inhaling.

Placing temperature sensors 36a, 36b at both sensor locations allows for benefits from both locations, but comes at a higher cost and complexity.

Referring now to FIG. 3, a portable liquid vaporizer 1 according to two different embodiments of the fourth aspect of the present invention is described next. The vaporizer 1 of the fourth aspect has many features in common with the vaporizer 1 of the first to third aspects, and each of the features and embodiments described in regard of the first to third aspects shall be understood to define corresponding features and embodiments of the fourth aspect. FIG. 3 shows a detail of the vaporizer 1 in a sectional view. The main difference between the embodiments is that the embodiment on the left comprises an insulation 14, which spaces the bottom of the heater 12 (more specifically, a central heating wire) apart from the surrounding plastic material 16 (so that the wire is isolated from direct contact with any plastic), whereas in the embodiment on the right, the heater 12 is surrounded by a thermally stable plastic such as PEEK or PCTG. Both embodiments withstand the temperatures needed for vaporizing cannabis concentrates. However, the insulation 14, being for example made from a ceramic, may provide an additional layer of protection for plastic parts in order to mitigate deformation and/or outgassing. The geometry and material of the insulation can vary. In addition to a ceramic, it can be made from a thermally stable plastic such as PEEK or PCTG.

Additional measures can be taken to further slow heat transfer such as constructing the air path tube from ceramic rather than metal, rerouting the air path 30 from the air inlet 71 through the reservoir 10 so that the oil contained therein is cooled. For instance, as shown in FIG. 4A, the air path 30 may enter the vaporizer 1 through an air inlet 71 contained in the housing 2, be routed through the reservoir 10, and then through the heater 12. However, as mentioned before, with the configuration disclosed herein, it is neither necessary to rely on such cooling effect nor necessary to use an insulation. Hence, the simplest design uses PEEK or PCTG as plastic material that comes into contact with the heater 12, and routes the air path 30 from an air inlet 71 that is present between the housing 2 and the bottom cap 4, or in the housing 2, directly towards the heater 12 without routing it through the reservoir 10, similar to the configurations shown in FIGS. 4B and 4C.

Next, a portable liquid vaporizer 1 according to an embodiment of the fifth aspect of the present invention is described with reference to FIG. 5. The vaporizer 1 of the fifth aspect has many features in common with the vaporizer 1 of the first to fourth aspects, and each of the features and embodiments described in regard of the first to fourth aspects shall be understood to define corresponding features and embodiments of the fifth aspect. Tamper proofing refers to the prevention of unauthorized oil filling into the pods without noticeable, significant damage to any part of the device and to the prevention of unauthorized pod use by the end user. A variety of methods can be considered to discourage tampering, which may be generally separated into three areas:

(a) Pod—Physical and Electronic Proofing

The pod may contain several one-way plastic snap fittings that resist disassembly without damage or permanent deformation to the soft plastic. These snap fittings are shown in FIG. 5. These snap fittings may also be covered so that they cannot be undone. FIG. 5A shows one-way plastic snap fittings (pod lower snap fittings 40a and pod upper snap fittings 40b) that hold the pod together.

Other methods may also include press fitting metal securing pins into the plastic parts, ultrasonic welding to join the plastics at a microscopic level, tamper tape, or even laser-engraving unique pod serial numbers. Batch serial numbers may be molded into the plastic which gives counterfeiters an additional level of complexity. Extreme tolerances required on the heater can be reflected in the battery checking the resistance of the heater as an actual or counterfeit device. At the mating interface between the pod and the battery, complex structures can be used to ensure only a specific mating orientation is allowed, which is then protected.

Moreover, as the pod may contain a PCB, additional electronic measures can be taken. Unique pod serial numbers can be printed onto the PCB substrate. Proprietary pogo pins/targets can be used. An EEPROM on the PCB is used to store oil data, but can also store retailer, manufacturer, and oil filler data. This data specifically is only a data string that, when connected to the battery and an APP, prompts the device to check online databases for the complete information. That connectivity is another layer of tamper resistance. The EEPROM may be the DS2431 from Maxim Integrated Products. With a 4×256 bit memory, there is more than enough space to store a variety of data strings or security keys. A QR code sticker can also be attached to the exterior of the pod (although QR codes are not as secure).

(b) Battery—Physical and Electronic Proofing

The internal componentry may be encased with extruded aluminium. Like the pod, the battery may also utilize one-way plastic snap fittings. Parts must be damaged and unique tools used to access the componentry. This does not pose an issue to repair teams who have enough replacement parts but provides a barrier for unauthorized manipulation.

Other methods may also be used to increase its tamper difficulty such as press-fit connections instead of screwed connections. Critical components such as the battery or PCB can be placed near likely locations of physical attack such that during the attack these critical components are irreversibly damaged. The PCB itself can be shielded or coated in an epoxy to resist examination or access using exposed traces. These methods do make repair highly difficult, thus necessitating a full replacement provided that any user-specific data such as historical data, favourites, and so on are stored in an online server rather than on the device itself.

(c) Supply Chain Proofing

The supply chain refers to the hardware manufacturers, the oil fillers, and the retailers. First or third party inspections of the hardware manufacturers may be possible depending on the contracts signed, unless those sectors are already vertically integrated with the business. These manufacturers would have their parts tested by labs in different countries such as China, USA, and Germany verifying the material and safety requirements. Test results may be published or shared with oil fillers and retails to further complicate counterfeiting and tampering efforts. The oil fillers would receive internet-connected filling devices that can both read-verify the pods and write relevant information to the pod. Finally, the retailers can also undergo first or third party inspections to ensure the entire product meets specifications before it reaches users' hands.

1. A Portable liquid vaporizer comprising:
a reservoir for holding a liquid to be vaporized;
a heater for vaporizing the liquid;
a mouthpiece for withdrawal of the vapor; and
an air path extending through the vaporizer, comprising a heating section and a cooling section, wherein the heating section extends along the heater and the cooling section extends directly downstream from the heater to the mouthpiece,
wherein the cooling section has an effective length that is sufficient for allowing the vapor to cool down and leave the mouthpiece at 40° C. or less above environmental temperature upon withdrawal of the vapor.

2. The portable liquid vaporizer of claim 1, wherein the cooling section has an effective length (L) that is at least 50 of the length (L) of the vaporizer and/or an effective length (L) that is at least 50 mm.

3. The portable liquid vaporizer of claim 1, wherein, when the mouthpiece defines the top, the heater is located in the lower half of the vaporizer.

4. The portable liquid vaporizer of claim 1, wherein a power source is arranged between the heater and the mouthpiece.

5. The portable liquid vaporizer of claim 1, wherein the longitudinal extension of the cooling section is offset from the longitudinal axis of the vaporizer.

6. The portable liquid vaporizer of claim 1, wherein the air path extends from the bottom of the vaporizer to the mouthpiece.

7. The portable liquid vaporizer of claim 1, wherein the air path extends through the reservoir upstream from the heating section.

8. A Portable liquid vaporizer comprising:
a reservoir for holding a liquid to be vaporized;
a heater for vaporizing the liquid;
a mouthpiece for withdrawal of the vapor; and
an air path extending through the vaporizer, comprising a heating section and a cooling section, wherein the heating section extends along the heater and the cooling section extends directly downstream from the heater to the mouthpiece,
wherein the cooling section comprises an insert for absorbing heat, filtering the vapor, catching droplets and/or flavoring the vapor.

9. The portable liquid vaporizer of claim 8, wherein the insert is configured to guide the vapor helically through the cooling section, and/or wherein the insert comprises a flavored body, preferably a flavored mesh-like or sponge-like body.

10. A portable liquid vaporizer comprising:
a reservoir for holding a liquid to be vaporized;
a heater for vaporizing the liquid;
a mouthpiece for withdrawal of the vapor;
an air path extending through the vaporizer, comprising a heating section and a cooling section, wherein the heating section extends along the heater and the cooling section extends directly downstream from the heater to the mouthpiece;
a control unit for controlling the heater; and
one or more temperature sensors configured to sense the temperature of the vaporized liquid in the cooling section,
wherein, when the one or more temperature sensors senses a temperature exceeding a predetermined threshold, the control unit is configured to reduce or stop the heating power of the heater.

11. The portable liquid vaporizer of claim 10, wherein the one or more temperature sensors are located in the cooling section adjacent to the heater and/or adjacent to the mouthpiece.

12. (canceled)

13. (canceled)

14. (canceled)

15. (canceled)

16. The portable liquid vaporizer of claim 10, wherein the control unit is configured to estimate a dosage of one or more active agents withdrawn from the vaporizer based on a mathematical model, wherein the mathematical model relates heating time, temperature of the heater, the flow rate and amount of the one or more active agents contained in the liquid to the dosage of the one or more active agents withdrawn from the vaporizer.

17. (canceled)

18. (canceled)

19. The portable liquid vaporizer of claim 10,
wherein the vaporizer is configured to prevent unauthorized manipulation thereof.

20. The portable liquid vaporizer of claim 10, wherein the reservoir is embedded in a tamper proof pod section so that the reservoir cannot be accessed without damage or permanent deformation to the pod section.

21. The portable liquid vaporizer of claim 10, wherein a power source is embedded in a tamper proof battery section so that the power source cannot be accessed without damage or permanent deformation to the battery section.

22. The portable liquid vaporizer of claim 10, wherein the vaporizer comprises a replaceable pod section and is configured to authenticate a certified pod section.

23. The portable liquid vaporizer of claim 22, wherein the authentication is based on verifying a data key stored on the pod section.

24. The portable liquid vaporizer of claim 10, wherein the vaporizer comprises a control unit for controlling the vaporizer, in particular the heater depending on data stored on the pod section.

25. (canceled)

26. The portable liquid vaporizer of claim 10, wherein the vaporizer is a pod-based or cartridge-based vaporizer.

27. (canceled)
